(19) United States
(12) Patent Application Publication
(10) Pub. No.: US 2015/0290227 A1
(43) Pub. Date: Oct. 15, 2015

(54) USE OF HAMAMELITANNIN FOR THE TREATMENT OF STAPHYLOCOCCAL INFECTIONS

(71) Applicant: Naomi Balaban, Hopkington, MA (US)

(72) Inventors: Naomi Balaban, Hopkinton, MA (US); Menachem Shoham, Richmond Heights, OH (US)

(21) Appl. No.: 14/747,977

(22) Filed: Jun. 23, 2015

Related U.S. Application Data
(63) Continuation of application No. 11/763,519, filed on Jun. 15, 2007.
(60) Provisional application No. 60/814,067, filed on Jun. 16, 2006.

(51) Int. Cl. A61K 31/7004; A61K 9/00

(52) U.S. Cl. CPC A61K 31/7004; A61K 9/0019

(57) ABSTRACT

RNAIII inhibiting peptide (RIP) has been established as an effective inhibitor of staphylococcal infections. Non-peptide small molecule analogs based on a pharmacophore conforming to the putative atomic structure of RIP, can be identified by computer screening of that pharmacophore against established libraries of known small molecules other than peptides. One such identified structural analog is hamamelitannin. When tested for effective inhibition of staphylococcal infections, hamamelitannin demonstrated inhibition similar to that exhibited by RIP. Other analogs can be identified and similarly used.

### CROSS-REFERENCE TO RELATED APPLICATIONS

This application is a continuation application of U.S. patent application Ser. No. 11/763,519, filed Jun. 15, 2007, now pending, which claims benefit of the filing date of provisional U.S. Patent Application 60/814,067, filed Jun. 16, 2006. The entire disclosure and contents of U.S. Ser. Nos. 60/814,067 and 11/763,519 are hereby incorporated by reference.

### STATEMENT OF GOVERNMENT FUNDING

Work on the invention disclosed and claimed herein may have been supported in part by federal funds pursuant to contract NIH 5R21AI059061-02. To that extent, the federal government may enjoy rights in this invention and the patents that protect it.

### BACKGROUND

1. Field of the Invention

The present invention relates generally to methods of addressing bacterial infection of mammals, and more particularly, infections in mammals, including but not limited to humans, of staphylococcal bacteria. Prominent and deadly infectious agents of this genus include Staphylococcus aureus and S. epidermidis.

The invention is directed to finding non-peptide molecules that inhibit these kinds of infections, in the same manner and fashion that RNAIII-Inhibiting Peptide (RIP) inhibits these infections. The non-peptide agents are identified by employing methods of structure-based drug design based on a pharmacophore deduced from the RIP sequence, and the crystal structure of related peptides. A pharmacophore is understood to be that set of structural features of a molecule that are responsible for that agent's activity. The useful agents are administered to mammals in need of treatment in the inhibition of staphylococcal bacterial infection, onto medical devices to prevent infections, or as additives to food or water.

2. Related Art

Staphylococcus aureus is a major human pathogen and is the most common cause of nosocornial pneumonia, surgical site and bloodstream infections, as well as community-acquired infections such as osteomyelitis and septic arthritis, skin infections, endocarditis, and meningitis. They cause such fatal diseases due to the expression of toxins like Toxic-shock syndrome toxin-1, enterotoxins, hemolysins, and other virulence factors that have been shown to affect the outcome of the infective process. The expression of virulence factors is highly regulated and involves cell-cell communication, otherwise known as quorum sensing.

There are two quorum-sensing systems that have so far been described in S. aureus and are referred herein as staphylococcal quorum sensing 1 & 2 (SQS 1 and SQS 2). A similar mechanism is operative in S. epidermidis and highly conserved throughout staphylococcal species.

SQS 1 consists of the autoinducer RNAIII-Activating Protein (RAP) and its target molecule RNAIII-Activating Protein (TRAP). At the mid-exponential phase of growth, SQS 1 induces the expression of SQS 2, which is encoded by the accessory gene regulator agr and is composed of agrABCD and hid (RNAIII). AgrD is a pro-peptide that yields an octapeptide pheromone (Autoinducing peptide, AIP) that is processed with the aid of AgrB. AgrC and AgrA are part of a bacterial two-component system, AgrC being the receptor component that is phosphorylated in an AIP ligand-dependent manner, and AgrA being a regulator. RNAIII is a polycistronic transcript, coding for delta hemolysin and acting as a regulatory RNA molecule that upregulates the expression of multiple exotoxins.

TRAP is a membrane associated 21 kDa protein that is histidine-phosphorylated, and its phosphorylation is necessary for activation of SQS 2 at the mid-exponential phase of growth. RAP is a 33 kDa protein that activates the agr by inducing the phosphorylation of TRAP. An antagonist of RAP, RNAIII-inhibiting peptide (RIP), inhibits the phosphorylation of TRAP and thereby strongly inhibits the downstream production of virulence factors, bacterial adhesion, biofilm formation, and infections in vivo. Upon disruption of the function of TRAP expression or phosphorylation, the bacteria lose their tendency to adhere and/or ability to form and maintain a biofilm, toxin expression level are reduced and in general, the development and worsening of bacterial related diseases is suppressed. Functional genomics studies indicate that in the absence of TRAP expression or phosphorylation (i.e., a TRAP phenotype), multiple virulence regulatory systems are disrupted, like the global regulatory locus agr (agrABCD and hid [RNAIII]), sarH2, otherwise known as sarU, which is a transcriptional activator of agr, and multiple virulence factors. These include alpha, beta, gamma and delta-hemolysin, triacylglycerol lipase precursor, glycerol ester hydrolase, hyaluronate lyase precursor, staphylococcal serine protease (V8 protease), cysteine protease precursor, cysteine protease, staphopain-cysteine proteinase, 1-phosphatidylinositol phosphodiesterase, zinc metalloprotease aureolysin precursor, holing-like proteins, and capsular polysaccharide synthesis enzymes. Clearly, TRAP belongs to a novel class of signal transducers. Thus, preventing TRAP expression or phosphorylation is a desired result as a means of inhibiting staphylococcal bacterial infections.

Patents reflecting the successful inhibition of staphylococcal bacterial infection, by using the inhibiting action, inter alia, of RIP include U.S. Pat. Nos. 7,067,135; 6,747,129 and 6,291,431, each of which is incorporated by reference. Pending U.S. patent applications Ser. Nos. 09/054,331 and 11/752,630 contain related disclosure, which is incorporated by reference as well. These patents and application discuss the use of peptide sequences (RIP) and antibodies to RAP, as well as antibodies to the target of RAP (TRAP), as means of preventing or inhibiting staphylococcal infection. Peptides present certain issues in telins of immune recognition, oral bioavailability, synthesis and purity. It would be desirable, and remains an object of those of skill in the art, to provide non-peptide chemical analogs.

### SUMMARY

According to a first broad aspect of the present invention, a method of identifying non-peptide analogs of RIP is provided, which comprises arriving at a molecular model of RIP, a pharmacophore, and applying it, based on distances between aromatic moieties, between hydrogen bond donors and acceptors, between aromatic moieties and those donors and acceptors, and applying it, through computer screening techniques, to databases of non-peptide candidates. Potential agents identified are considered for compatibility with therapeutic and pharmacological constraints, and then tested, in vitro and in vivo for ability to inhibit infection. Successful inhibition identifies a molecule that may be used to prevent and treat staphylococcal infections, particularly those caused by S. aureus and S. epidermidis.

According to a second broad aspect of the invention, there is provided a composition comprising one or more of the successfully identified non-peptide RIP analogs. One of these analogs is hamamelitannin. Other analogs are, similarly identified as a therapeutic pharmaceutical treatment.

A third aspect of the invention is the administration of the effective non-peptide analogs of RIP discussed herein to a mammal, or inhibit staphylococcal bacterial infection, especially those caused by S. aureus and S. epidermidis and thereby reduce or prevent the same. Administration can be through any conventional means or through application to devices such as implants, catheters, tampons and bandages, which are in turn applied to the mammalian patient.

### BRIEF DESCRIPTION OF THE DRAWINGS

The invention will be described in conjunction with the accompanying drawings, in which:

FIG. 1 is a view of molecular model of RIP prepared on the basis of homology with the crystal structure of a related protein, ribosomal protein L2 within the crystal structure of the 50S ribosomal subunit from the bacterium Deinococcus radiodurans (Protein Data Bank code 1NKW);

FIG. 2 is a representation of the pharmacophore used in the invention to identify potential nonpeptide inhibitory compounds with distances presented in Å units;

FIG. 3 is a Representation of the Chemical Structure of Hamamelitannin.

FIG. 4 is a graphical presentation of Hamamelitannin inhibition of RNAIII production: 2×10⁷ early exponential S. aureus cells containing rnaiii::blaZ fusion construct were grown for 2.5 hrs with increasing amounts of hamamelitannin or RIP. RNAIII levels were determined as β-lactamase activity (reporter gene product) and denoted as Vₘₐₓ.

FIG. 5 demonstrates graphically the inhibition of S. aureus attachment in vitro by hamamelitannin: S. aureus cells were placed in polystyrene plates and incubated with increasing amounts of hamamelitannin or RIP for 3 hrs at 37° C. without shaking. Attached cells were stained and _{OD595nm} was determined.

FIG. 6 is a graph presentation of the effectiveness of bacterial graft-associated infection inhibition by hamamelitannin and RIP against challenge by methicillin resistant S. aureus and S. epidermidis (MRSA and MRSE) using pre-incubation.

FIG. 7 is a graph presentation of the effectiveness of bacterial graft-associated infection inhibition by hamamelitannin against challenge by MRSA and MRSE using local treatment (graft soak).

### DETAILED DESCRIPTION

### Model Building of the RIP Peptide

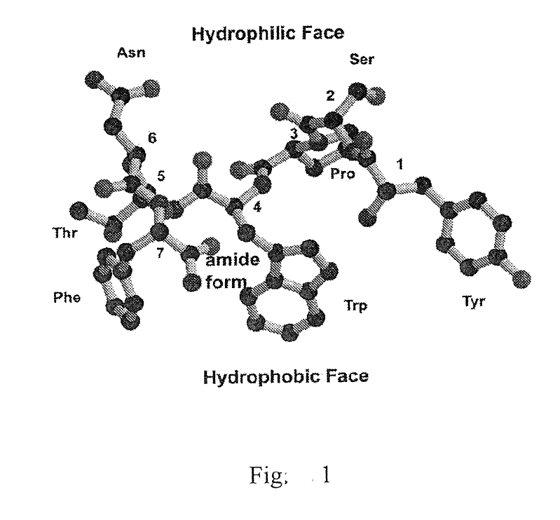

Short peptides such as RIP do not have a fixed conformation in solution. However, the active conformation of RIP can be deduced from the corresponding sequence segment in RAP since RIP competes with RAP for binding to the same receptor. This suggests that RIP is structurally similar to a segment of RAP and that probably RAP acts as an agonist and RIP as an antagonist of RAP. The sequence of RIP (YSPWTNF) is similar to the sequence of residues 4-9 of RAP (YKPITN). Consequently, it is reasonable to assume that the structure of RIP is very similar to the corresponding segment in RAP, if not entirely identical. Therefore, it would be best to build a model of RIP based on the corresponding segment in the RAP structure. However, a crystal structure or a solution NMR structure of RAP is not available. Fortunately, there is another way to build a model of the active conformation of RIP. A crystal structure of a protein similar to RAP is available. This is the crystal structure of ribosomal protein L2 from Deinococcus radiodurans (PDB code 1 NKW). The structure of protein L2 from S. aureus is expected to be very similar to the structure of L2 from Deinococcus radiodurans because L2 is a highly conserved protein. A model of RIP was built based on the crystal structure of ribosomal protein L2 from Deinococcus radiodurans (PDB code 1 NKW). This homology-built model of RIP was subjected to energy minimization with program CNS (FIG. 1).

Amino acid residues 1, 3 and 5 in RIP are entirely conserved, and in residues 2 and 4 the sequence differences are conservative, i.e. an arginine instead of a serine and a tyrosine instead of a tryptophan. RIP homologs with such conservative amino acid replacements in these positions are known to retain their inhibitory activity.

In silico Screening for RIP Analogs

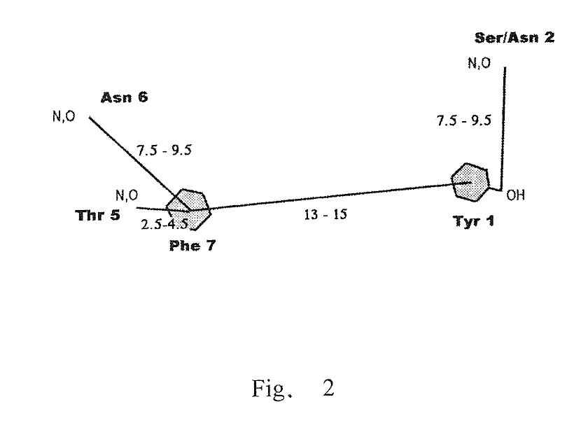

Screening for small molecule nonpeptide analogs of RIP was carried out by a computer search with the ISIS software (Integrated Scientific Information System) from Elsevier MDL against the Available Chemicals Database (ACD), a library of 300,000 commercially available small molecule compounds. The model of RIP served as the basis for the search. Our first approach was to carry out similarity searches with the RIP models against the ACD. As this search yielded only peptides it was abandoned. Next we turned to a search of the ACD based on a pharmacophore (FIG. 2).

### Definition of a Pharmacophore for a Nonpeptide RIP Analog

The basis for the pharmacophore design was the RIP model. The pharmacophore was defined in terms of distances in the RIP model between aromatic moieties, distances between aromatic moieties and hydrogen donors or acceptors and distances between pairs of hydrogen bond donors/acceptors. Different pharmacophores were used in the search for a suitable RIP analog. FIG. 2 shows the pharmacophore that led to the identification of hamamelitannin as an inhibitor of staphylococcal infections. Distances are indicated in units of Angstroms (Å). This pharmacophore calls for distances of 13-15 Å between the centers of two aromatic rings corresponding to Tyr 1 and Phe 7; distances of 7.5-9.5 Å between the hydroxyl group of Tyr 1 and a hydrogen donor or acceptor corresponding to Ser 2 or Asn 2, consisting of a nitrogen or oxygen atom; distances of 7.5-9.5 Å between the center of the aromatic ring corresponding to Phe 7 and a hydrogen donor or acceptor corresponding to Asn 6, consisting of a nitrogen or oxygen atom; distances of 2.5-4.5 Å between the center of an aromatic ring corresponding to Phe 7 and a hydrogen donor or acceptor corresponding to Thr 5, consisting of a nitrogen or oxygen atom.

### Hamamelitannin Inhibits RNAIII Production in Vitro.

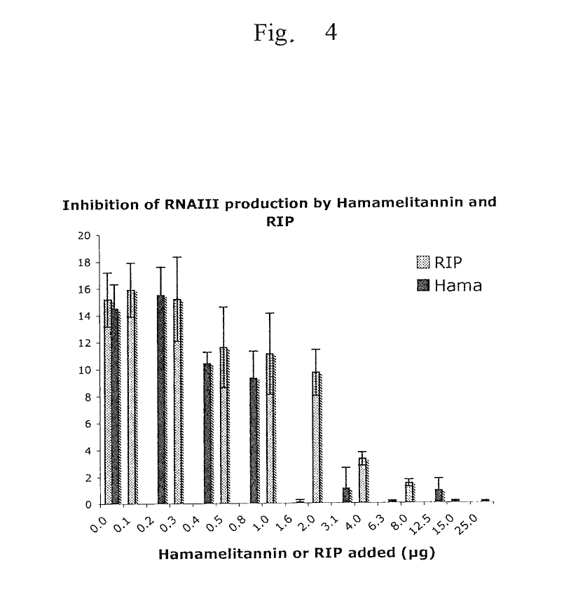

To test if hamamelitannin is a quorum sensing inhibitor and thus suppresses RNAIII synthesis, 2×10⁷ cells were incubated with increasing amounts (0-50 μg) of hamamelitannin (or RIP). RNAIII levels were measured as β-lactamase activity as a reporter gene product by the addition of nitrocefin as substrate. As shown in FIG. 4, hamamelitannin inhibits RNAIII synthesis in a dose dependent manner, and is most effective at doses >2 μg/2×10⁷ bacteria (2 nanomoles/10⁷ bacteria or 0.2 picomoles/10³ bacteria). RIP also inhibited RNAIII production in a dose dependent manner, and was most effective in doses of 8 μg/2×10⁷ bacteria (0.5 picomoles/10³ bacteria). Hamamelitannin (2′,5-di-O-galloyl hamamelose) was purchased from Chromadex, Inc. (93% purity, as assessed by HPLC by the manufacturer). Hamamelitannin was dissolved in water at 25 mg/ml and stored at −70° C. until use.

### Hamamelitannin Inhibits Cell Attachment in Vitro

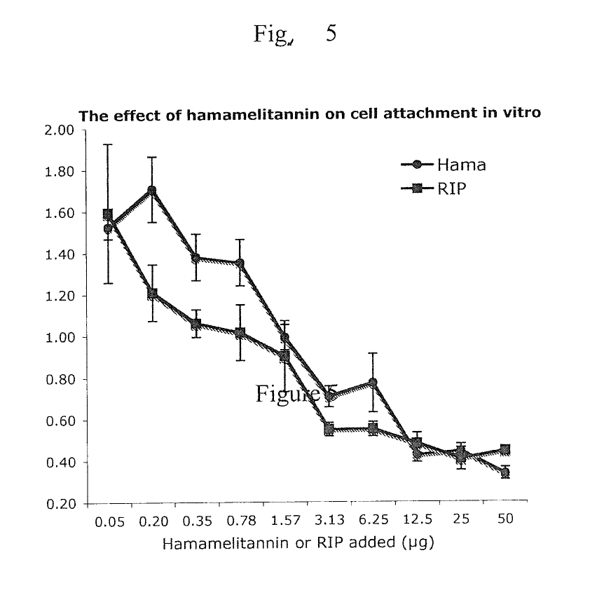

To test for the effect of hamamelitannin on bacterial attachment in vitro, S. aureus cells were incubated with 0-50 μg hamamelitannin or RIP in polystyrene plates for 3 hrs at 37° C. Adherent bacteria were stained with methylene blue and OD determined at 595 nm. As shown in FIG. 5, hamamelitannin (or RIP) reduced cell attachment in a dose-dependent manner and were most effective when 2×10⁷ bacteria were grown in the presence of at least 12.5 μg hamamelitannin or RIP (0.4 picomoles/10³ bacteria). Similar results were obtained with S. epidermidis.

Coating with Hamamelitannin Prevents Device-Associated Infections in Vivo in a Rat Model

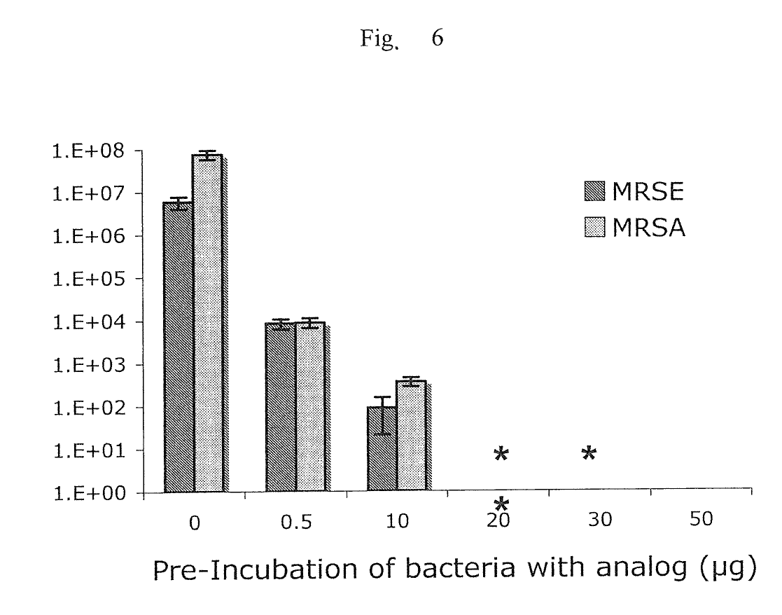

To measure the amount of hamamelitannin necessary to prevent device-associated infections, bacteria (2×10⁷ MRSA or MRSE) were pre-incubated with increasing amounts of hamamelitannin for 30 min at room temperature. Grafts were implanted and rats were challenged with the pre-incubated bacteria. Seven days later the graft was removed and bacterial load determined. As shown in FIG. 6, bacterial load on the graft decreased with increasing dose of hamamelitannin while bacterial load in the control untreated group was ˜10⁷ CFU/ml. No bacteria was found when either MRSA or MRSE were pre-incubated with >20 μg hamamelitannin.

In the testing reflected in FIG. 6 and summarized above, to ascertain the amount of hamamelitannin/bacteria necessary to potentially prevent device-associated infections, bacteria (2×10⁷ MRSE or MRSA in 150 μl saline) were pre-incubated with hamamelitannin (0, 0.5, 10, 20, 30, 50 μg) for 30 min at room temperature, as noted. Grafts were implanted and rats were challenged with the pre-incubated bacteria. Seven days later the graft was removed and bacterial load determined. As shown in FIG. 6, all 10 control rats included in the untreated control group (without pre-treatment) demonstrated evidence of graft infection, with quantitative culture results showing 5.9×10⁶±1.9×10⁶ colony-forming units (CFU)/ml MRSE. When the bacteria was treated with 0.5, 10, 20, 30 or 50 μg hamamelitannin, MRSE load was 8.4×10³±2.2×10³ for 0.5 μg and 8.7×10¹±6.6×10¹ for 10 μg. No sign of infection was found when bacteria were pre-incubated with 20, 30 or 50 μg hamamelitannin. Similar results were obtained for MRSA, where untreated control group (without pre-treatment) demonstrated evidence of graft infection, with quantitative culture results showing 7.6×10⁷±1.9×10⁷ CFU/ml MRSA, while those treated with 0.5 or 10 μg hamamelitannin only showed 8.8×10³±2.3×10³ and 3.6×10²±0.8×10² CFU/mL, respectively. No sign of infection was detected if bacteria were pre-incubated with 20, 30 or 50 μg hamamelitannin. These results indicate that 10 μg hamamelitannin could prevent an infection caused by 2×10⁷ CFU MRSE or MRSA, which is what was found for RIP.

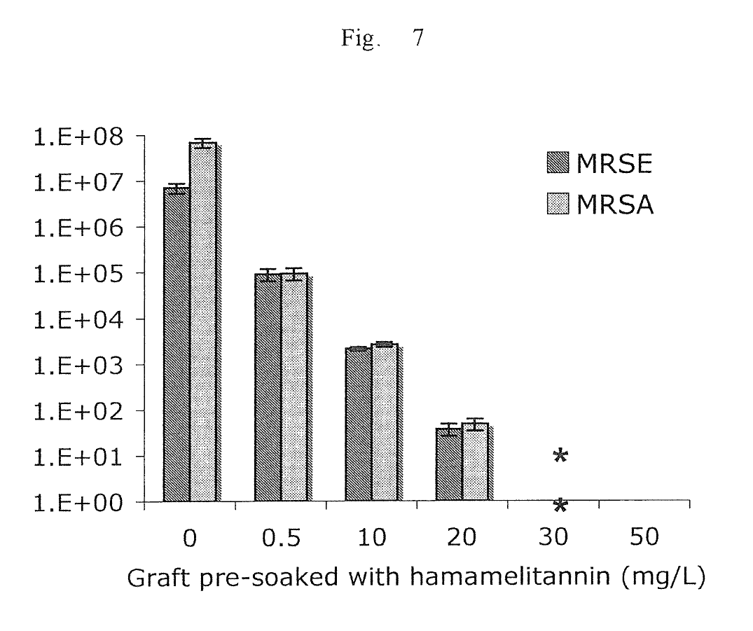

To test if hamamelitannin could inhibit graft-associated infection, grafts (1 cm² collagen coated Dacron) were soaked for 1 h in increasing hamamelitannin concentrations. The graft was then implanted into the animal, and bacteria injected onto the graft. Seven days later the graft was removed and bacteria on the graft counted. All rats included in the untreated control groups demonstrated evidence of graft infections, with quantitative culture results showing 7.0×10⁶±1.7×10⁶ CFU/ml MRSE and 6.8×10⁷±1.5×10⁷ CFU/ml MRSA. Significant (p<0.05) decrease in bacterial load was found when the grafts were presoaked with hamamelitannin (0-50 mg/L). As shown in FIG. 7, animals challenged with MRSE and grafts pre-soaked with 0.5 mg/L hamamelitannin had 9.0×10⁴±2.6×10⁴ CFU/mL, those soaked with 10 mg/L hamamelitannin had 2.1×10³±0.2×10³ CFU/mL, those soaked with 20 mg/L had 3.7×10′±1.1×10¹ CFU/mL, and those soaked with 30 or 50 mg/L had no sign of bacterial load and thus had <10 CFU/mL. Very similar results were obtained when animals were challenged with MRSA. Specifically, as shown in FIG. 7, grafts soaked with 0.5, 10, 20, 30 or 50 mg/L hamamelitannin had bacterial load of 9.3×10⁴±2.8×10⁴, 2.6×10³±0.3×10³, 3.7×10¹±1.1×10¹ CFU/mL, respectively and no bacterial load was detected when the graft was pre-soaked in 30 or 50 mg/L hamamelitannin.

As shown, the analog identification method of the invention presents a powerful new tool for identifying agents for the treatment and prevention of virulent staphylococcal infections. RIP has been previously shown to act as a powerful agent suppressing virulence in staphylococcal infections, likely through suppression of normal responses to quorum sensing. Hamamelitannin exhibits similar inhibitory activity. Similar mechanisms may well be in play, in light of the inhibition of RNAIII production shown.

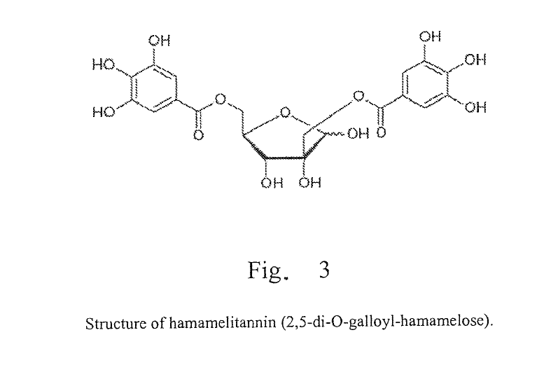

Hamamelitannin (2,5-di-O-galloyl-hamamelose) is an ester of hamamelose (2-hydroxymethyl-D-ribose) with two molecules of gallic acid (FIG. 3). Since gallic acid contains three phenolic functional groups, hamamelitannin is considered a polyphenol. It belongs to the family of tannins, which are plant polyphenols that are used in tanning animal hides into leather.

Hamamelitannin is a natural product found in the bark and the leaves of Hamamelis virginiana (witch hazel), a deciduous shrub native to damp woods in eastern North America and Canada. The concentration of hamamelitannin in the bark and the leaves is 5 and less than 0.04% (w/w), respectively. Witch hazel extracts were used by Native Americans for pain relief, colds and fever, and they are currently used in skin care products and in dermatological treatment of sun burn, irritated skin, atopic eczema as well as to promote wound healing via anti-inflammatory effects. Hamamelitannin also was shown to inhibit tumor necrosis factor α-mediated endothelial cell death at concentrations less than 100 uM. Hamamelitannin, at a minimum concentration of 50 uM, was also found to have a high protective activity against cell damage induced by peroxides or UVB radiation. In addition, some antibacterial properties of witch hazel have been reported, where aqueous extracts of the bark or the leaves inhibited the growth of E. coli, S. aureus, B. subtilis and E. faecalis. In contrast, we have determined that hamamelitannin has no effect on bacterial growth in vitro even at concentrations as high as 2.5 mM per 1000 bacteria, 13,000 times the MIC (minimum inhibitory concentration) of ampicillin to the same S. aureus strain (0.2 uM per 1000 bacteria).

Hamamelitannin and RIP inhibit RNAIII production at minimal doses of 0.2 and 0.5 picomoles per 1000 bacteria, respectively. Hamamelitannin and RIP also inhibit cell attachment in vitro, both at doses equal or greater than 0.4 picomoles per 1000 bacteria.

Hamamelitannin presents a non-peptide small molecule alternative to RIP as an excellent inhibitor of device-associated infections in vivo, in line with its inhibitory effect on RNAIII and cell attachment in vitro. Inhibition of infection is concentration dependent. Grafts pre-soaked with at least 30 mg/L (˜60 uM) hamamelitannin, show no signs of infection even though the animals were challenged with a high bacterial load of 2×10⁷ CPU. These results are similar to those observed with RIP. Device-associated infections are prevented by merely soaking a graft in the hamamelitannin solutions, suggesting that hamamelitannin can be used to coat medical devices to prevent staphylococcal infections, including those caused by drug resistant strains MRSA and MRSE.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless specifically and expressly excluded thereby.

What is claimed is:

1. A method of inhibiting Staphylococcal infection in a mammal, comprising administering, to a source of said staphylococcal infections, an amount of a composition comprising a non-peptide small molecule analog of RNAIII-Inhibiting peptide (RIP) which inhibits Staphylococcus infections in a mammal, wherein said analog is present in an amount effective to inhibit said Staphylococcus infection in said mammal, said composition further comprising a pharmaceutically acceptable carrier.

2. The method of claim 1, wherein the Staphylococcus is S. aureus.

3. The method of claim 1, wherein said composition comprises, as a RIP analog, hamamelitannin.

4. The method of claim 1, wherein said administering comprises applying said composition to an exposed surface of a device to be inserted into said mammal's body.

5. The method of claim 4, wherein said device is selected from the group consisting of an implantable device, a catheter, a tampon and a bandage.
